(19) United States
(12) Patent Application Publication
(10) Pub. No.: US 2013/0245221 A1
(43) Pub. Date: Sep. 19, 2013

(54) PROCESS FOR PREPARING ETHYLENE AND PROPYLENE

(71) Applicants: Leslie Andrew CHEWTER, Amsterdam (NL); Sivakumar SADASIVAN VIJAYAKUMARI, Amsterdam (NL); Jeroen VAN WESTRENEN, Amsterdam (NL)

(72) Inventors: Leslie Andrew CHEWTER, Amsterdam (NL); Sivakumar SADASIVAN VIJAYAKUMARI, Amsterdam (NL); Jeroen VAN WESTRENEN, Amsterdam (NL)

(73) Assignee: SHELL OIL COMPANY, Houston, TX (US)

(21) Appl. No.: 13/606,284

(22) Filed: Sep. 7, 2012

(30) Foreign Application Priority Data
Sep. 7, 2011 (EP) 11180334.2

(51) Int. Cl. C07C 1/22; C07C 29/00; C07C 2/12; C07C 4/06; C07C 2/00; C08F 110/02; C07D 301/02

(52) U.S. Cl. CPC C07C 1/22; C08F 110/02; C07C 29/00; C07D 301/02; C07C 4/06; C07C 2/00; C07C 2/12
USPC 526/352; 568/852; 549/523; 585/640; 585/315; 585/324; 585/319; 585/329

(57) ABSTRACT

The present invention provides a process preparing ethylene and propylene, comprising the step of:
a) contacting a feed comprising a tert alkyl ether obtained from an etherification reaction between ethanol and a tertiary iso-olefin with a zeolite-comprising catalyst at a temperature in the range of from 350 to 1000° C. to obtain a olefinic product comprising ethylene and propylene.

This application claims the benefit of European Application No. 11180334.2 filed Sep. 7, 2011, which is incorporated herein by reference.

### FIELD OF THE INVENTION

The invention relates to a process for preparing ethylene and propylene.

### BACKGROUND TO THE INVENTION

Methanol-to-olefin processes are well described in the art. Typically, methanol-to-olefin processes are used to produce predominantly ethylene and propylene. An example of such a methanol-to-olefin process is described in WO-A 2006/020083. In the process of WO-A 2006/020083, the methanol is first converted into dimethylether (DME) prior to be subjected to a conversion to olefins, thereby reducing the amount of water produced during the conversion to olefins. Both methanol and DME are suitable feedstocks for a Methanol-to-olefin process and therefore such processes are also generally referred to as oxygenate-to-olefin (OTO) processes.

Ethanol can also be converted in to ethylene. U.S. Pat. No. 4,207,424 describes a process wherein ethanol is converted to ethylene in a dehydration reaction using an alumina catalyst. U.S. Pat. No. 4,727,214 describes a process wherein ethanol is converted to ethylene using a crystalline zeolitic catalyst. Other ethanol dehydration processes have been described extensively in the prior art. A disadvantage of these processes is that only ethylene is formed in a ethanol dehydration process, where methanol-to-olefin processes convert methanol or DME to a product slate containing both ethylene and propylene. Ethanol is mentioned, among numerous other oxygenates, in several prior art documents, including for instance US20090105429, US20090187058, US20100298619, US20090187059, US20090187057, US20090187056 as an optional feedstock to an oxygenate to olefins process. However none of these documents describe the conversion of ethanol in any detail, nor do they provide any information on the expected product slate.

In EP2108637, a two step process is proposed to convert ethanol into ethylene and propylene. In the process of EP2108637, ethanol is dehydrated in a first process step. The ethanol is dehydrated to ethylene by contacting the ethanol with a silicate or zeolitic catalyst. In a subsequent process step the obtained ethylene is provided together with a C4+ fraction, for instance obtained from an FCC, to an olefin cracking process (OCP), wherein part of the ethylene is converted with the C4+ olefins to propylene.

In the example described in EP2108637, ethylene, produced by a dehydration of an ethanol-comprising feed, is combined with a C4 cut of a FCC to form a feedstock comprising ethylene C4 olefins and C4 paraffins. The latter feedstock is subjected to an olefin cracking process to convert at least part of the ethylene and C4 olefins to propylene. The obtained effluent of the OCP reactor contained ethylene and propylene at a weight ratio of approximately 2 to 1 and a C4 hydrocarbon fraction. The C4 hydrocarbon fraction comprises approximately 50wt % olefins and 50Wt % paraffins and can be recycled back to the OCP reactor.

A disadvantage of the process as described in EP2108637 is that substantial amounts of inert paraffins are provided to the OCP reactor together with the required C4 olefins. When recycling the C4 hydrocarbon fraction in the effluent of the OCP, these paraffins are also recycled leading to a built up of paraffins in the feed to the OCP.

There is a need in the art to produce olefinic products from ethanol-based feedstocks wherein the amount of paraffins provided to the process is reduced.

### SUMMARY OF THE INVENTION

It has now been found that it is possible to reduce the amount of paraffins provided to an oxygenate conversion process by providing an tert-alkyl ether-comprising feedstock, wherein the tert alkyl ether is obtained from an etherification reaction between ethanol and a tertiary iso-olefin

Accordingly, the present invention provides a process for preparing ethylene and propylene, comprising the step of:
a) contacting a feed comprising a tert alkyl ether obtained from an etherification reaction between ethanol and a tertiary iso-olefin with a zeolite-comprising catalyst at a temperature in the range of from 350 to 1000° C. to obtain a olefinic product comprising ethylene and propylene.

The process according to the present invention has the advantage that propylene may be produced from ethanol via an intermediate etherification step with a tertiary iso-olefin. The obtained tert-alkyl ether can be converted to ethylene and propylene in a single step process.

By providing tertiary iso-olefins to the process as part of the tert-alkyl ether, less C4+ olefins need to be provided separately to the process and consequently the amount of paraffins provided to the process together with the C4+ olefins is reduced.

Furthermore, the process according to the present invention may produce an olefinic product comprising ethylene and propylene at a weight ratio close to 1, compared to the typically ethylene-rich olefinic products obtained in the prior art.

Additionally, the process allows for extraction of iso-olefins from a C4+ olefin fraction. The iso-olefins are provided as part of the feed to the process according to the invention to produce the olefinic product, while the remainder may be used for further purposes.

### DETAILED DESCRIPTION OF THE INVENTION

Ethanol may suitably be used as part of the feed to an Oxygenate-to-Olefin (OTO) process to produce ethylene and/or propylene, i.e. an olefinic product comprising ethylene and propylene. In the process according to the present invention ethanol is first converted into a tert-alkyl ether by an etherification reaction with an tertiary iso-olefin. The tert-alkyl ether obtained by an etherification reaction of ethanol with a tertiary iso-olefin is herein also referred to as an ethyl tert-alkyl ether or ETAE, preferably ethanol is converted into an ethyl tert-butyl ether (ETBE) by an etherification reaction with isobutene. The obtained ETAE is subsequently converted to an olefinic product comprising ethylene and propylene by providing a feed comprising the ETAE. The ETAE in the feed is converted to the olefinic product by contacting the feed with a zeolite-comprising catalyst at a temperature in the range of from 350 to 1000° C. Preferably, the olefinic product comprises advantageously at least 50 mol %, in particular at least 50 wt %, ethylene and propylene, based on total hydrocarbon content in the olefinic product.

In the process according to the invention the olefinic product comprises ethylene and propylene, preferably in a weight ratio of ethylene to propylene in the range of from 0.5 to 1.5. This has the advantage that the product slate of the olefinic product is more balanced and less additional ethylene is produced in case of an increase in the propylene demand.

Zeolite-comprising catalyst suitable for converting the ETAE-comprising feedstock preferably include zeolite-comprising catalyst compositions. Such zeolite-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Zeolites preferably have a molecular framework of one, preferably two or more [SiO₄] and [AlO₄] tetrahedral units. These silicon and aluminum based zeolites have been described in detail in numerous publications including for example, U.S. Pat. No. 4,567,029. In a preferred embodiment, the zeolites have 8-, 10- or 12-ring structures and an average pore size in the range of from about 3 Å to 15 Å.

Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Zeolite-comprising catalysts have the advantage that in addition to the conversion of methanol or ethanol, these catalysts also induce the conversion of tert-alkyl ethers.

Preferred catalysts comprise a more-dimensional zeolite, in particular of the MFI type, more in particular ZSM-5, or of the MEL type, such as zeolite ZSM-11. A zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. Zeolite-comprising catalyst that comprise a more-dimensional zeolite of the MFI type and/or MEL type, preferably ZSM-5 and/or ZSM-11, more preferably ZSM-5, have the particular advantage that they can convert ETAE, in particular ETBE, to an olefinic product comprising ethylene and propylene at a weight ratio close to 1, preferably in the range of from 0.5 to 1.5, more preferably 0.8 to 1.1, even more preferably 0.9 to 1.0.

A preferred MFI-type and/or MEL-type, more preferably MFI-type zeolite, even more preferably ZSM-5, has a Silica-to-Alumina ratio (SAR) of in the range of from 40 to 100, preferably of from 50 to 90. The lower silica to alumina ratio has the advantage that a high yield of ethylene and propylene may be obtained when converting ETAE, in particular ETBE.

Particular preferred catalysts include catalysts comprising one or more zeolites having one-dimensional 10-membered ring channels, i.e. one-dimensional 10-membered ring channels, which are not intersected by other channels. Preferred examples are zeolites of the MTT and/or TON type. Preferably, the catalyst comprises at least 40wt %, preferably at least 50% wt of such zeolites based on total zeolites in the catalyst.

Preferably, the catalyst comprises at least one zeolite selected from ZSM-22 and ZSM-23 zeolites. Contrary to ZSM-5 and ZSM-11, which are multi dimensional zeolites, these are zeolites having one-dimensional 10-membered ring channels, which are known for their particular suitability to convert oxygenates.

The catalyst may comprise phosphorus, as such or in a compound, i.e. phosphorus other than any phosphorus included in the framework of the molecular sieve. It is preferred that the ZSM-5 and/or ZSM-11 zeolite comprises additionally phosphorus. The phosphorus may be introduced by pre-treating the zeolite prior to formulating the catalyst and/or by post-treating the formulated catalyst comprising the MEL or MFI-type zeolites. Preferably, the catalyst comprises phosphorus as such or in a compound in an elemental amount of from 0.05-10 wt % based on the weight of the formulated catalyst. A particularly preferred catalyst comprises phosphor-treated ZSM-5 having SAR of in the range of from 50 to 90, more preferably of from 80 to 90.

It is preferred that the zeolites in the hydrogen form are used, e.g. HZSM-5, HZSM-22, and HZSM-23. Preferably at least 50wt %, more preferably at least 90wt %, still more preferably at least 95wt % and most preferably 100w % of the total amount of zeolite used is in the hydrogen form. It is well known in the art how to produce such zeolites in the hydrogen form.

The olefinic product comprises ethylene and propylene, but may also comprise other components, in particular C4+ olefins. Preferably, C4+ olefins are separated from the olefinic product as part of a C4+ hydrocarbon fraction and provided at least in part to step (a) of the process to form at least part of the feed to the process. Preferably, the C4+ hydrocarbon fraction comprises in the range of from 50 to 100 wt % of C4 and C5 olefins, based on the olefins in the C4+ hydrocarbon fraction, more preferably of from 50 to 100 wt % of C4 olefins, based on the olefins in the C4+ hydrocarbon fraction.

The C4+ hydrocarbon fraction comprises both normal olefins and iso-olefins, in particular tertiary iso-olefins. In one embodiment of the invention at least part of the C4+ olefins are provided to step (a) by subjecting at least part of the C4+ olefins in the C4+ hydrocarbon fraction to an etherification process with ethanol to obtain a ETAE, preferably including ethyl tert-butyl ether. ETAE is subsequently separated from the remainder of the C4+ hydrocarbon fraction and provided at least in part to step (a).

In the process according to the invention, C4+ iso-olefins may extracted from the C4+ hydrocarbon fraction by reacting iso-olefins with ethanol to form ETAE, such as ethyl tert-butyl ether (ETBE) and tert-amyl ethyl ether (TAEE). Preferably, the tert-alkyl ether is ethyl tert-butyl ether. The formed ethers can be separated from the remainder of the C4+ hydrocarbon fraction.

Only iso-olefins, wherein the double bound is located directly adjacent to a tertiary carbon atom can react with methanol to form tert-alkyl ethers. Such iso-olefins are herein referred to as tertiary iso-olefins. Examples of such tertiary iso-olefins include isobutene, 2-methyl-1-butene and 2-methyl-2-butene. An example of an iso-olefin that is not a tertiary iso-olefin is 3-methyl-1-butene. The C4+ fraction contains at least tertiary iso-olefins.

In the etherification process the C4+ hydrocarbon fraction is contacted with ethanol, in the presence of a suitable etherification catalyst. When the iso-olefins, preferably isobutylene, 2-methyl-1-butene or 2-methyl-2-butene in the hydrocarbon stream are contacted with the ethanol in the presence of an etherification catalyst, at least part of the iso-olefins are converted with the alcohol to tert-alkyl ethers. Reference herein in to a tert-alkyl ether is to an ether of an alcohol and a tertiary iso-olefin. From the etherification process, an etherification product stream is retrieved. The etherification product stream will comprise the formed tert-alkyl ethers and the remainder of the hydrocarbon stream, i.e. the unreacted components, including C4+ normal-olefins and optionally other hydrocarbons. In addition, the etherification product stream may also comprise unreacted ethanol. Typically the etherification reaction is performed in the presence of an excess of alcohol, i.e. above reaction stoichiometry with the iso-olefin.

At least part, and preferably all, of the etherification product stream is separated into at least an ether-enriched stream and an iso-olefin-depleted C4+ hydrocarbon fraction, including C4+ normal olefins and optionally other hydrocarbons. The separation of the etherification product stream into an ether-enriched stream and an iso-olefin-depleted hydrocarbon stream can be done with normal separation means provided in the art. Due to the relatively high boiling points of ethanol, the bulk of the excess of alcohol can be directed toward the ether-enriched stream.

The iso-olefin-depleted C4+ fraction may be exported from the process and used for other purposes, such as for instance part of a raffinate-2 stream.

Alternatively, at least part of the iso-olefin-depleted C4+ fraction may also be provided to step (a), although it is preferred that part of the iso-olefin-depleted C4+ hydrocarbon fraction is withdrawn from the process as a purge stream. The advantage of withdrawing part of the iso-olefin-depleted C4+ hydrocarbon fraction is that the build-up of paraffins and other hydrocarbon components in the feed to step (a) is reduced. Paraffins present in the C4+ hydrocarbon fraction are not converted when contacted with the catalyst in step (a). By first extracting iso-olefins from the C4+ hydrocarbon fraction, the concentration of olefins in the C4+ hydrocarbon fraction is reduced, and consequently less valuable olefins are lost as part of the purge. An additional advantage of providing at least part of the iso-olefins-depleted C4+ hydrocarbon fraction to step (a) of the process, is that this fraction may still comprise some ethanol, which is a valuable feed for producing further ethylene and propylene.

At least part of the C4+ olefins in the C4+ hydrocarbon fraction may also be provided to a further step (b) and converted to a further olefinic product comprising ethylene and propylene in a process generally referred to as an olefin cracking process (OCP) using a zeolite-comprising catalyst. Preferably, the C4+ olefins in the C4+ hydrocarbon fraction are contacted with the zeolite-comprising catalyst at a temperature in the range of from 350 to 1000° C., preferably 350 to 750° C.; and a pressure from 0.1 kPa (1 mbar) to 5 MPa (50 bar), preferably from 100 kPa (1 bar) to 1.5 MPa (15 bar). Optionally, the stream comprising C4+ olefins provided to the OCP also contains or is provided together with a diluent. Examples of suitable diluents include, but are not limited to, such as water or steam, nitrogen, argon and methane.

Any zeolite-comprising catalyst suitable for cracking olefins may be used. Preferably, the zeolite-comprising catalyst of step (b) is the same as the zeolite-comprising catalyst in step (a). By using the same catalyst for both step (a) and step (b) the catalyst regeneration facilities may be shared.

In the process according to the invention, a feed comprising ETAE is provided to step (a). The ETAE may be any ether obtained from an etherification ratio between ethanol and a tertiary iso-olefin. Preferably, the feed comprises at least one of ETBE or TAEE.

The ETAE may have been obtained from any source. The ETAE may be obtained by reacting an etherification process of ethanol with a tertiary iso-olefin, wherein the tertiary iso-olefin is provided externally, for instance as part of an external C4+ hydrocarbon fraction. However, the tertiary iso-olefin may also be provided as part of an internal stream, such as the effluent of the step (a) or the optional OCP process of step (b). In this way at least part of a C4+ hydrocarbon fraction, i.e. at least part of the tertiary iso-olefins, from the effluent of the step (a) or the optional OCP process of step (b) may be recycled to step (a). Examples of external hydrocarbon streams are the C4 and C5 fractions of the effluent of a refinery unit such as thermal cracking units, catalytic cracking units, steam cracking units, naphtha (steam) cracking units, butadiene extraction units and semi-hydrogenation units for removal of C4 and C5 diolefins. A particularly preferred C4 hydrocarbon stream is raffinate-1.

Preferably, at least 70 wt %, preferably 90wt % of the ETAE is, during normal operation, provided by reacting ethanol with tertiary iso-olefins contained in a C4+ hydrocarbon fraction in the effluent from step (a) and optionally step (b), based on the ETAE provided to step (a).

It preferred that at least 70 wt %, preferably 90wt % of the C4 iso-olefins, i.e. isobutene, in the effluent of step (a), based on the C4 iso-olefins in the effluent of step (a) are, during normal operation, provided back to step (a) preferably as ETBE. More preferably, at least 70 wt %, preferably 90wt % of all tertiary iso-olefins in the effluent of step (a), based on all tertiary iso-olefins in the effluent of step (a) are, during normal operation, provided back to step (a) preferably as ETAE.

A particular advantage of the process according to the invention is that olefins, i.e. tertiary iso-olefins, are isolated from a C4+ hydrocarbon fraction and provided to step (a) of the process according to the invention in the form of ETAE. Whereas, any inert paraffins also present C4+ hydrocarbon fraction advantageously remain in now iso-olefins-depleted C4+ hydrocarbon fraction instead of being co-fed to process.

The feed to step (a) of process according to the invention may comprises other oxygenates. The feed provided to step (a) of the process may comprise methanol. An advantage of using a feed that comprises in addition to ETAE also methanol is that irrespective of the nature of the zeolite in the catalyst, an olefinic product may be obtained comprising ethylene and propylene at a weight ratio close to 1, preferably in the range of from 0.5 to 1.5, more preferably 0.8 to 1.1, even more preferably 0.9 to 1.0.

Where the feed comprises methanol and an ETAE, the weight ratio of methanol to ETAE in the feed is preferably in the range of from 0.15 to 20, preferably 0.25 to 15.

The methanol and ethanol, i.e. the ethanol used to produce the ETAE, are preferably bio-methanol and/or bio-ethanol. The use of bio-methanol and/or bio-ethanol contributes in reducing the carbon dioxide footprint of the process.

The feed to step (a) may also comprise C4+ olefins. The C4+ olefins may for instance include comprising C4+ normal olefins and iso-olefins, for example 1-butene, 2-butene, isobutene, 1-pentene and/or 2-pentene, 2-methyl-1-butene or 2-methyl-2-butene.

When the ETAE is the only oxygenate and no other reactive components such as olefins are present in the feed, the feed, by nature of the ETAE, has a theoretical molar ratio of oxygenate to olefin of 1:1. In case further oxygenates and/or olefins are provided to step (a) as part of the feed comprising ETAE, the preferred molar ratio of oxygenate to olefin in the feed to step (a) lies in the range of 20:1 to 1:10, more preferably in the range of 18:1 to 1:5, still more preferably in the range of 15:1 to 1:3, even still more preferably in the range of 12:1 to 1:3. For purposes of calculating the molar ratio of oxygenate to olefin in the total feed, the olefins provided to the process as part of the ethyl tert-alkyl ether must also be taken into account.

The feed to step (a) of the process can comprise an amount of diluents. Examples of suitable diluents include, but are not limited to, such as water or steam, nitrogen, argon and methane. Preferably steam or water is used as the diluent.

A variety of oxygenate-to-olefin (OTO) processes is known for converting oxygenates to an olefin-containing product, as already referred to above. One such process is described in WO-A 2006/020083. Processes integrating the production of oxygenates from synthesis gas and their conversion to light olefins are described in US20070203380A1 and US20070155999A1.

The reaction conditions of step (a) include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

Typically the catalyst deactivates in the course of the process, primarily due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the zeolite. This may also apply to the catalyst used in optional step (b) of the process. Conventional catalyst regeneration techniques can be employed to remove the coke.

The catalyst particles used in the process of the present invention can have any shape known to the skilled person to be suitable for this purpose, for example it can be present in the form of spray dried catalyst particles, spheres, tablets, rings, extrudates, etc. Extruded catalysts can be applied in various shapes, such as, cylinders and trilobes. If desired, spent catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

Both the OTO process of step (a) as the optional OCP process of step (b) may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred.

In the process according to the invention iso-olefins may be reacted with ethanol in an etherification process. The etherification process may be any suitable etherification process available in the art for etherifying ethanol and iso-olefins to tert-alkyl ethers. Reference is made to the Handbook of MTBE and Other Gasoline Oxygenates, H. Hamid and M. A. Ali ed., 1^{st} edition, Marcel Dekker, New York, 2004, pages 65 to 223, where several established process and catalyst for preparing tert-alkyl ethers such as ETBE are described. In particular reference is made to chapter 9, pages 203 to 220 of the Handbook of MTBE and Other Gasoline Oxygenates, wherein suitable commercial etherification processes are described. A preferred etherification process is an etherification process wherein the iso-olefins are converted with ethanol to a tert-alkyl ether in the presence of a catalyst. Any homogeneous or heterogeneous Brönsted acid may be used to catalyze the etherification reaction. Such catalyst include: sulfuric acid, zeolites, pillared silicates, supported fluorocarbonsulphonic acid polymers and protonated cation-exchange resins catalyst, preferred catalyst are protonated cation-exchange resins catalyst due to the higher catalytic activity and the bound acid sites. A commonly used catalyst is Amberlyst 15.

Preferably, the iso-olefins are converted with ethanol, to a tert-alkyl ether at a temperature in the range of from 30 to 100° C., more preferably 40 to 80° C. Preferably, the iso-olefins are converted with ethanol to a tert-alkyl ether at a pressures in the range of from 5 to 25 bar, more preferably 6 to 20 bar.

In step (a) of the process an olefinic product comprising ethylene and propylene is retrieved. As described herein above, in case of step (b) a further olefinic product comprising ethylene and propylene may be obtained. The ethylene and propylene may be separated from the remainder of the components in the olefinic products. Preferably, the olefinic product and further olefinic product are at least partially, and preferably fully, combined prior to separating the ethylene and propylene from the remaining components. The ethylene may be further converted into at least one of polyethylene, mono-ethylene-glycol, ethylbenzene and styrene monomer. The propylene may be further converted into at least one of polypropylene and propylene oxide.

### EXAMPLES

The invention is illustrated by the following non-limiting examples.

### Example 1

Several zeolite catalysts were tested to show their ability to convert ETBE-comprising feedstocks to ethylene and propylene.

Powders of the respective zeolites were pressed into tablets and the tablets were broken into pieces and sieved. For the catalytic testing, the sieve fraction of 60-80 mesh was used.

Prior to reaction, the catalyst was treated ex-situ in air at 600° C. for 2 hours.

The reaction was performed using a quartz reactor tube of 1.8 mm internal diameter. The catalyst samples were heated in nitrogen to 525° C. and a feed mixture consisting of 6 vol % of reactants balanced in N₂ was passed over the catalyst at atmospheric pressure (1 bar). Two different feed mixtures were used:
(I) a feed mixture comprising 6 vol % of ETBE;
(II) a feed mixture comprising 3 vol % of ETBE and 3 vol % of methanol.

The Gas Hourly Space Velocity (GHSV) is determined by the total gas flow over the catalyst weight per unit time (ml.g_{catalyst}⁻¹.h⁻¹). The gas hourly space velocity used in the experiments was 24,000 (ml.g_{catalyst}⁻¹.h⁻¹).

The effluent from the reactor was analyzed by gas chromatography (GC) to determine the product composition.

The composition has been calculated on a weight basis of all hydrocarbons analyzed.

The results are shown in Table 1.

TABLE 1

| | | | | | | | | Yield | Ratio |
|---|---|---|---|---|---|---|---|---|---|
| | | C2= | C3= | C4 | C5 | C6+ | Light ends | C2= + C3= | C2=/C3= |
| Feed | Catalyst | [wt %] | [wt %] | [wt %] | [wt %]] | [wt %] | [wt %] | [wt %] | [—] |
| I | ZSM-5* | 35.5 | 37.2 | 16.3 | 2.6 | 8.3 | 0.1 | 73 | 0.95 |
| II | ZSM-5* | 31.3 | 31.9 | 20.2 | 3.5 | 12.7 | 0.3 | 63 | 0.98 |
| I | ZSM-5^{#} | 33.7 | 27.5 | 28.0 | 3.5 | 6.9 | 0.3 | 61 | 1.22 |
| II | ZSM-5^{#} | 31.8 | 34.0 | 20.3 | 3.7 | 8.9 | 1.2 | 66 | 0.93 |
| I | ZSM-23 | 35.3 | 16.5 | 44.2 | 1.8 | 2.1 | 0.1 | 52 | 2.13 |
| II | ZSM-23 | 31.7 | 32.9 | 27.2 | 3.9 | 4.0 | 0.2 | 65 | 0.96 |
| I | ZSM-22 | 34.4 | 11.8 | 50.4 | 1.6 | 1.6 | 0.1 | 46 | 2.87 |
| II | ZSM-22 | 30.7 | 32.9 | 29.3 | 3.7 | 3.2 | 0.2 | 64 | 0.93 |

*SAR 80
^{#}SAR 280

For all tested catalyst, the oxygenate conversion was complete. No ETBE, ethanol or methanol was detected in the effluent of the reactor.

As can be seen from Table 1, when a ZSM-5 zeolite is used as the catalyst, ETBE is converted to an olefinic product with a high yield of ethylene and propylene. In addition, the ratio of ethylene to propylene in the olefinic product is close to 1, indicating that a substantial part of the ETBE has been converted to propylene. Lowering the SAR of the ZSM-5 from 280 to 80, results in an increase of both the propylene yield as well as the combined ethylene and propylene yield.

By co-feeding methanol, an olefinic product is obtained with an ethylene to propylene ratio below 1 for all catalyst. Co-feeding methanol in case of the ZSM-22 and ZSM-23 not only results in an improved propylene yield, also the combined ethylene and propylene yield is significantly improved. The effect of co-feeding is less significant for the ZSM-5 catalyst. It may be concluded that, based on the results in table 1, it is possible to add ETBE to an existing methanol-based OTO process, without significant implications for the obtained ethylene and propylene yields and ethylene to propylene ratio.

Comparative Example A (not according to the invention)

Using a procedure similar to Example 1, the same zeolites were tested using methyl tert-butyl ether (MTBE) instead of ETBE. The feed mixtures used were:

i) a feed mixture comprising 6 vol % of MTBE;

ii) a feed mixture comprising 3 vol % of MTBE and 3 vol % of methanol.

The results are shown in Table 2.

TABLE 2

| | | | | | | | | Yield | Ratio |
|---|---|---|---|---|---|---|---|---|---|
| | | C2= | C3= | C4 | C5 | C6+ | Light ends | C2= + C3= | C2=/C3= |
| Feed | Catalyst | [wt %] | [wt %] | [wt %] | [wt %] | [wt %] | [wt %] | [wt %] | [—] |
| i | ZSM-5* | 26.8 | 38.1 | 19.4 | 2.7 | 13.0 | 0.0 | 65 | 0.70 |
| ii | ZSM-5* | 25.7 | 37.6 | 18.9 | 3.2 | 13.8 | 0.7 | 63 | 0.68 |
| i | ZSM-5^{#} | 17.9 | 39.8 | 27.6 | 3.2 | 9.7 | 1.8 | 58 | 0.45 |
| ii | ZSM-5^{#} | 17.7 | 42.4 | 21.9 | 3.3 | 12.9 | 1.8 | 60 | 0.42 |
| i | ZSM-23 | 20.7 | 42.9 | 30.1 | 2.0 | 3.6 | 0.6 | 64 | 0.48 |
| ii | ZSM-23 | 21.5 | 46.7 | 22.2 | 2.8 | 6.2 | 0.8 | 68 | 0.46 |
| i | ZSM-22 | 17.2 | 39.9 | 36.4 | 2.1 | 3.9 | 0.4 | 64 | 0.43 |
| ii | ZSM-22 | 17.8 | 48.5 | 25.0 | 2.6 | 5.2 | 0.8 | 66 | 0.37 |

*SAR 80
^{#}SAR 280

It will be clear from table 2 that by replacing ETBE by MTBE, a much lower ratio of ethylene to propylene is obtained consistently both for the feed comprising MTBE alone and in combination with a methanol co-feed. In addition, in particular for ZSM-5-comprising catalyst the combined yield of ethylene and propylene is reduced when replacing ETBE by MTBE.

What is claimed is:

1. A process for preparing ethylene and propylene, comprising the step of:
a) contacting a feed comprising a tert alkyl ether obtained from an etherification reaction between ethanol and a tertiary iso-olefin with a zeolite-comprising catalyst at a temperature in the range of from 350 to 1000° C. to obtain a olefinic product comprising ethylene and propylene.

2. A process according to claim 1, wherein the olefinic product comprises ethylene and propylene in a weight ratio of ethylene to propylene in the range of from 0.5 to 1.5.

3. A process according to claim 1, wherein the feed comprises ethyl tert-butyl ether

4. A process according to claim 1, wherein the zeolite-comprising catalyst comprises ZSM-5.

5. A process according to claim 4, wherein the ZSM-5 has a silica to alumina ratio of in the range of from 50 to 90.

6. A process according to claim 1, wherein the catalyst further comprises at least one zeolite selected from TON-type and MTT-type zeolites.

7. A process according to claim 1, wherein the olefinic product further comprises C4+ olefins and at least part of the C4+ olefins are provided to step (a) as part of the feed.

8. A process according to claim 7, wherein the at least part of the C4+ olefins are provided to step (a) by subjecting at least part of the C4+ olefins to an etherification process with ethanol to obtain a tert-alkyl ether, preferably including ethyl tert-butyl ether, and providing at a least part of the tert-alkyl ether to step (a).

9. A process according to claim 7, wherein iso-olefin-depleted C4+ fraction of the olefinic product is exported from the process and used for other purposes.

10. A process according to claim 8, wherein iso-olefin-depleted C4+ fraction of the olefinic product is exported from the process and used for other purposes.

11. A process according to claim 1, wherein the olefinic product further comprises C4+ olefins and at least part of the C4+ olefins are provided to a further step (b) and converted to a further olefinic product comprising ethylene and propylene in an olefin cracking process using a zeolite-comprising catalyst.

12. A process according to claim 11, wherein the catalyst of step (b) is the same as the catalyst in step (a).

13. A process according to claim 1, wherein feed comprises methanol and the weight ratio of methanol to tert-alkyl ether in the feed is in the range of from 0.15 to 201.

14. A process according to claim 1, wherein the ethylene is further converted into at least one of polyethylene, mono-ethylene-glycol, ethylbenzene and styrene monomer.

15. A process according to claim 1, wherein the propylene is further converted into at least one of polypropylene and propylene oxide.
